(19) United States
(12) Patent Application Publication
(10) Pub. No.: US 2022/0403322 A1
(43) Pub. Date: Dec. 22, 2022

(54) CO-CULTURE METHOD FOR BIOFUEL AND BIOCHEMICAL PRODUCTION FROM UNTREATED SYNGAS

(71) Applicant: Northeastern University, Boston, MA (US)

(72) Inventors: Benjamin Woolston, Stoneham, MA (US); Anthony Stohr, Richmond, RI (US)

(21) Appl. No.: 17/718,927

(22) Filed: Apr. 12, 2022

Related U.S. Application Data
(60) Provisional application No. 63/173,756, filed on Apr. 12, 2021.

(51) Int. Cl. C12N 1/20; C12P 7/52

(52) U.S. Cl. CPC C12N 1/20; C12P 7/52; C12N 2500/02

(57) ABSTRACT

Disclosed are methods related to culturing anaerobic bacteria in a microaerobic environment. The method comprises culturing in a microaerobic environment an anaerobic bacteria with an aerobic microorganism. The microaerobic environment may not require gas pre-treatment to remove trace O₂. Also disclosed are methods related to producing a product, syngas fermentation, and gas valorization. The method comprises culturing in a microaerobic environment an anaerobic bacteria with an aerobic microorganism.

### RELATED APPLICATION

This application claims the benefit of priority to U.S. Provisional Patent Application Ser. No. 63/173,756, filed Apr. 12, 2021.

### BACKGROUND

Acetogens are obligate anaerobic bacteria that dwell in energy-poor environments near the very thermodynamic limit of life, making a living by using hydrogen (H₂), carbon monoxide (CO), and other reduced organic carbon compounds to convert carbon dioxide (CO₂) into acetate. To compete in these resource-constrained environments, they have evolved one of nature's most efficient pathways for CO₂ reduction, the Wood-Ljungdahl (WL) pathway. In addition to acetate, some acetogens also produce fuel-like molecules including ethanol, as well as higher-value molecules such as the polymer precursor 2,3-butanediol (2,3-BDO). The high energetic efficiency of the WL pathway, and the ability of acetogens to grow on a wide range of substrates—including renewable H₂, CO-rich waste industrial gases and biomass-derived synthesis gas—has made them attractive microbes for the renewable production of biofuels and biochemicals. Commercial demonstrations are underway; most notably LanzaTech (Skokie, Ill.), who have scaled up an acetogenic process for producing ethanol from steel mill off-gas at 90,000 metric tons per year, and recently demonstrated high-productivity syngas fermentation to acetone and isopropanol. Despite the promise of gas fermentation, there remain significant technological challenges hindering widespread adoption: First, acetogenic bacteria are strict anaerobes, necessitating costly gas pre-treatment to remove trace O₂ before fermentation. Second, as anaerobes, acetogens are energy-limited, and therefore only capable of producing low-margin compounds like acetate and ethanol at industrially relevant levels. Overcoming these challenges could enable a dramatic expansion in the adoption of gas fermentation as part of the 21^{st} century renewable bioeconomy.

### SUMMARY

Disclosed are methods of culturing anaerobic. The method comprises culturing in a microaerobic environment the anaerobic bacteria and an aerobic microorganism. Numerous embodiments are further provided that can be applied to any aspect of the present invention described herein. For example, in some embodiments, the microaerobic environment does not require gas pre-treatment to remove trace O₂. In some embodiments, the microaerobic environment comprises O₂, CO, H₂, and CO₂; O₂, N₂, H₂, and CO₂; O₂, CH₄, H₂, CO₂, and CO; or O₂, CH₃OH, CHOOH, and CO₂.

In some embodiments, the aerobic microorganism consumes oxygen in the culture to establish low-oxygen conditions for growth of the anaerobic bacteria. In some embodiments, the anaerobic bacteria are acetogenic bacteria. In some embodiments, the acetogenic bacteria convert CO, H₂, and CO₂ to acetate. In some embodiments, the acetogenic bacteria are of the genus Clostridium, Moorella, Ruminococcus, Acetobacterium, Eubacterium, Butyribacterium, Blautia, Alkalibaculum, Sporomusa, or Oxobacter. In some embodiments, the acetogenic bacteria are Clostridium ljungdahlii, Clostridium autoethanogenum, Eubacterium limosumi, Acetobacterium woodii, Butyribacterium methylotrophicum, Blautia hydrogenotrophica, Clostridium carboxidivorans, Clostridium drakei, Clostridium coskatii, Alkalibaculum bacchi, Blautia product, Sporomusa ovata, Sporomusa silvacetica, Sporomusa sphaeroides, Oxobacter pfennigii, Clostridium aceticum, Clostridium formicaceticum, Clostridium tetanomorphum, Moorella thermoacetica, Moorella thermoautotrophica, Ruminococcus productus, or Acetobacterium woodii.

In some embodiments, the aerobic microorganism is an aerobic acetotroph microorganism. In some embodiments, the aerobic acetotroph microorganism converts the acetate to a product. In some embodiments, the product is ethanol, 2,3-butanediol (2,3-BDO), bioproducts 3-hydroxybutyrate (3HB), butanol, farnesene, acetic acid, butyric acid, methane, or an amino acid. In some embodiments, the amino acid is aspartate.

In some embodiments, the aerobic microorganism is Escherichia coli, Vibrio natriegens, or Yarrowia lipolytica. In some embodiments, the aerobic microorganism is of the genus Azotobacter. In some embodiments, the aerobic microorganism is Azotobacter vinelandii or Azotobacter chroococcum. In some embodiments, the aerobic microorganism is a methanotroph microorganism. In some embodiments, the aerobic microorganism is a Type II methanotroph bacteria. In some embodiments, the methanotroph bacteria are of the genus Methylocystis. In some embodiments, the methanotroph bacteria are Methylocystis bryophila.

In another aspect, disclosed are methods of producing a product. The method comprises culturing in a microaerobic environment an anaerobic bacteria and an aerobic microorganism. In another aspect, disclosed are methods of syngas fermentation. The method comprises culturing in a microaerobic environment an anaerobic bacteria and an aerobic microorganism. In another aspect, disclosed are methods of gas valorization. The method comprises culturing in a microaerobic environment an anaerobic bacteria and an aerobic microorganism.

Numerous embodiments are further provided that can be applied to any aspect of the present invention described herein. For example, in some embodiments, the microaerobic environment does not require gas pre-treatment to remove trace O₂. In some embodiments, the microaerobic environment comprises O₂, CO, H₂, and CO₂; O₂, N₂, H₂, and CO₂; O₂, CH₄, H₂, CO₂, and CO; or O₂, CHOOH, CHOOH, and CO₂.

In some embodiments, the aerobic microorganism consumes oxygen in the culture to establish low-oxygen conditions for growth of the anaerobic bacteria. In some embodiments, the anaerobic bacteria are acetogenic bacteria. In some embodiments, the acetogenic bacteria convert CO, H₂, and CO₂ to acetate. In some embodiments, the acetogenic bacteria are of the genus Clostridium, Moorella, Ruminococcus, Acetobacterium, Eubacterium, Butyribacterium, Blautia, Alkalibaculum, Sporomusa, or Oxobacter. In some embodiments, the acetogenic bacteria are Clostridium ljungdahlii, Clostridium autoethanogenum, Eubacterium limosumi, Acetobacterium woodii, Butyribacterium methylotrophicum, Blautia hydrogenotrophica, Clostridium carboxidivorans, Clostridium drakei, Clostridium coskatii, Alkalibaculum bacchi, Blautia product, Sporomusa ovata, Sporomusa silvacetica, Sporomusa sphaeroides, Oxobacter pfennigii, Clostridium aceticum, Clostridium formicaceticum, Clostridium tetanomorphum, Moorella thermoacetica, Moorella thermoautotrophica, Ruminococcus productus, or Acetobacterium woodii.

In some embodiments, the aerobic microorganism is an aerobic acetotroph microorganism. In some embodiments, the aerobic acetotroph microorganism converts the acetate to a product. In some embodiments, the product is ethanol, 2,3-butanediol (2,3-BDO), bioproducts 3-hydroxybutyrate (3HB), butanol, farnesene, acetic acid, butyric acid, methane, or an amino acid. In some embodiments, the amino acid is aspartate.

In some embodiments, the aerobic microorganism is Escherichia coli, Vibrio natriegens, or Yarrowia lipolytica. In some embodiments, the aerobic microorganism is of the genus Azotobacter. In some embodiments, the aerobic microorganism is Azotobacter vinelandii or Azotobacter chroococcum. In some embodiments, the aerobic microorganism is a methanotroph microorganism. In some embodiments, the aerobic microorganism is a Type II methanotroph bacteria. In some embodiments, the methanotroph bacteria are of the genus Methylocystis. In some embodiments, the methanotroph bacteria are Methylocystis bryophila.

### BRIEF DESCRIPTION OF THE DRAWINGS

FIG. 1 shows “microaerobic” co-culture approach for producing high-value bioproducts from CO₂-rich oxygen-containing gas feeds.

FIG. 2 shows predicted production envelopes (product yield vs growth rate) for co-culture (light) vs. acetogen monoculture (dark) for the bioproducts 3-hydroxybutyrate (3HB) and 2,3-butanediol (2,3-BDO) as well as the biofuels butanol and farnesene. For all these high-value products, the co-culture is predicted to provide superior performance to an engineered acetogen.

FIG. 3 shows reported growth of aerobes and anaerobes on the same scale. E. coli can grow aerobically at dissolved oxygen (DO) concentrations much lower than have been found to enable anaerobic growth of several strict anaerobes, suggesting a wide range of DO that could support ‘microaerobic’ co-culture.

FIGS. 4A and 4B show co-culture growth and production. FIG. 4A shows co-culture growth (dark=E. coli, light=C. ljungdahlii, Black=redox) in rich medium sparged with 1% O₂. FIG. 4B shows production in rich medium sparged with 1% O₂.

FIG. 5 shows successful co-culture growth (Dark=E. coli, Light=C. ljungdahlii) in minimal medium sparged with 2% O₂.

FIG. 6 shows successful co-culture growth (Dark=E. coli, Light=C. ljungdahlii) with reverse inoculation strategy and higher oxygen content in sparge gas.

FIG. 7 shows nitrogen-fixing co-culture for high-efficiency fertilizer/amino acid production from CO₂, H₂, and N₂.

FIG. 8 shows bioplastics from waste plastics via co-culture: an acetogen/methanotroph co-culture for waste plastic upcycling.

FIG. 9 shows that acetogens are used in the microaerobic co-culture to enable production of higher-value molecules from an expanded set of C1 substrates.

### DETAILED DESCRIPTION

This is the first evidence of the sustained growth of an acetogen in a microaerobic environment. A microaerobic environment is an environment where the dissolved oxygen concentration in the growth media is substantially reduced compared to the equilibrium concentration in normal atmosphere.

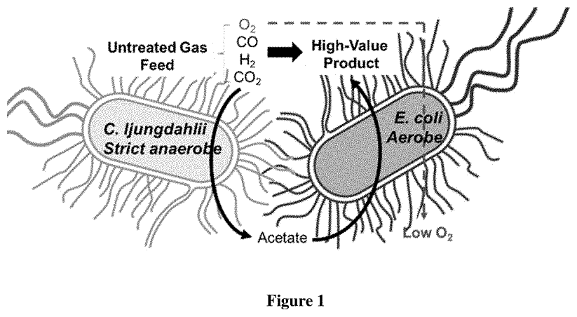

Despite the promise of gas fermentation, there remain significant technological challenges hindering widespread adoption: First, acetogenic bacteria are strict anaerobes, necessitating costly gas pre-treatment to remove trace O₂ before fermentation. Second, as anaerobes, acetogens are energy-limited, and therefore only capable of producing low-margin compounds like acetate and ethanol at industrially relevant levels. The disclosed method is a novel synthetic co-culture approach that solves both these problems simultaneously. In this system (FIG. 1), gas fermentation and product formation are split between two different microbes in a single reactor. The acetogen, such as Clostridium ljungdahlii, converts CO, H₂, and CO₂ to acetate at high yield. A partner organism, Escherichia coli, is responsible for converting the acetate to a higher-value product, and for rapidly consuming the oxygen in the reactor to establish the low-oxygen (nano-molar, or ‘microaerobic’) conditions necessary for C. ljungdahlii growth. In essence, the E. coli “protects” C. ljungdahlii from oxygen in exchange for fixed carbon.

There are several important advantages to this approach for gas fermentation:

(1) Because this is a mutualistic co-culture (i.e. each microbe is dependent on the other for survival), stability of the consortium is ensured, compared to other synthetic co-cultures where additional control strategies are required.

(2) E. coli has been shown to grow aerobically at oxygen concentrations as low as 3 nM. This makes it an ideal partner for scavenging O₂, while reaping the energetic benefits of aerobic respiration for product synthesis.

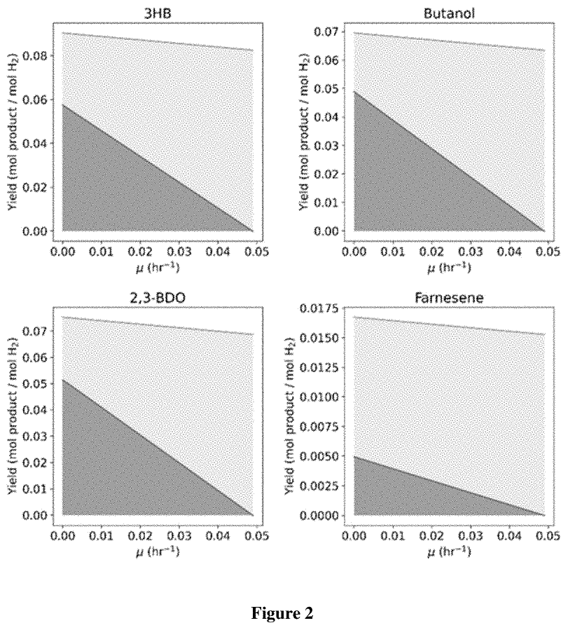

(3) Our production envelope calculations suggest that, for a wide range of products, the co-culture could outperform an engineered acetogenic mono-culture, offering equally high yields at a significantly higher growth rate (FIG. 2). There is a particularly noticeable increase in performance for products which have a direct requirement for ATP in the metabolic pathway.

(4) Scalability and safety: Our preliminary calculations (Table 1) suggest that, in a H₂-limited chemostat with modest mass transfer, the co-culture could produce the bioproduct succinate from CO₂ and H₂ at a productivity of 4.6 g L⁻¹ h⁻¹ with a nonflammable gas feed (O_{2=1.6}%). By contrast, the safety of typical aerobic H₂ fermentations with hydrogenotrophs such as Cupriavidus necator has been a major limitation to commercialization due to their high demand for O₂. The lower demand for O₂ in our system reflects the high ATP efficiency of the WL pathway for CO₂ fixation, compared to the energy intensive Calvin cycle.

(5) The co-culture can be implemented in a single reactor, reducing technical complexity compared to other solutions that rely on multi-reactor systems separating CO₂ fixation and product synthesis.

TABLE 1
Predicted performance metrics for production of succinate (as an example)
from CO₂/H₂ using microaerobic co-culture, based on reported monoculture performance
metrics. The co-culture can reach high productivities using a non-explosive gas mixture,
providing a significant benefit over existing technologies.

| Parameter | Definition | Value | Source |
|---|---|---|---|
| k_{L}a | Gas mass transfer coefficient | 800 h⁻¹ | Hu, 2016 |
| Y_{H}_{₂} | Mol fraction of H₂ in gas feed | 0.7 | Experience |
| P | Reactor pressure | 1 atm | Experience |
| ${k}_{{H}_{{H}_{2}}}$ | Henry's constant for H₂ | 7.8 x 10⁻⁴ mol L⁻¹ atm⁻¹ | NIST |
| ${k}_{{H}_{{O}_{2}}}$ | Henry's constant for O₂ | 1.3 x 10⁻³ mol L⁻¹ atm⁻¹ | NIST |
| D | Dilution rate | 0.075 h⁻¹ | Mock, 2014 |
| ${Y}_{\frac{Ac}{{H}_{2}}}$ | Acetate yield (C. ljungdahlii) | 0.23 mol acetate mol H₂⁻¹ | Fast, 2012 |
| q_{H}_{₂} | Specific hydrogen uptake rate | 0.07 mol H₂ gDW⁻¹ h⁻¹ | Richter, 2013 |
| q_{Ac} | Specific acetate uptake rate (E. coli) | 0.026 mol acetate gDW⁻¹ h⁻¹ | Kutscha, 2020 |
| q_{O}_{₂} | Specific oxygen uptake rate for | 0.005 mol O₂ gDW⁻¹ h⁻¹ | Varma, 1993 |
| | aerobic growth at D = 0.075 h⁻¹ | | |
| ${Y}_{\frac{Succ}{Ac}}$ | Maintenance-adjusted succinate yield (E. coli) | 0.447 mol succinate mol acetate⁻¹ | FBA simulations |
| Output | Definition | Value | Equation |
| X_{Cl} | C. ljungdahlii biomass | 6.3 g L⁻¹ | $\frac{{k}_{L} a \cdot {y}_{{H}_{2}} \cdot P}{{q}_{{H}_{2}}}$ |
| X_{Ec} | E. coli biomass | 3.4 L⁻¹ | $\frac{{k}_{L} a \cdot {y}_{{H}_{2}} \cdot P \cdot {Y}_{\frac{Ac}{{H}_{2}}}}{{q}_{Ac}}$ |
| Q_{succ} | Production rate of succinate | 4.6 g L⁻¹ h⁻¹ | ${k}_{L} a \cdot {y}_{{H}_{2}} \cdot P \cdot {Y}_{\frac{Ac}{{H}_{2}}} \cdot {Y}_{\frac{Succ}{Ac}}$ |
| ${y}_{{O}_{{2}_{min}}}$ | Minimum mol fraction of O₂ in gas feed | 0.016 (1.6%) | $\frac{{q}_{{O}_{2}} \cdot {X}_{Ec}}{{k}_{L} a \cdot P \cdot {k}_{{H}_{{O}_{2}}}}$ |

Syngas fermentation, also known as synthesis gas fermentation, is a microbial process. In this process, a mixture of hydrogen, carbon monoxide, and carbon dioxide, known as syngas, is used as carbon and energy sources, and then converted into fuel and chemicals by microorganisms. The main products of syngas fermentation include ethanol, butanol, acetic acid, butyric acid, and methane. Certain industrial processes, such as petroleum refining, steel milling, and methods for producing carbon black, coke, ammonia, and methanol, discharge enormous amounts of waste gases containing mainly CO and H₂ into the atmosphere either directly or through combustion. Biocatalysts can be exploited to convert these waste gases to chemicals and fuels as, for example, ethanol.

There are several microorganisms, which can produce fuels and chemicals by syngas utilization. These microorganisms are mostly known as acetogens including Clostridium ljungdahlii, Clostridium autoethanogenum, Eubacterium limosum, Clostridium carboxidivorans P7, Peptostreptococcus productus, and Butyribacterium methylotrophicum. Most use the Wood-Ljungdahl pathway.

The concept of co-culture engineering has gained increasing attention in the last few years, but to our knowledge, an aerobic co-culture has never been used for syngas fermentation, and no one has demonstrated growth of an acetogenic microbe with sustained oxygen sparging. We have demonstrated that E. coli and C. ljungdahlii can be co-cultivated in a bioreactor sparged with a gas mix containing oxygen. More broadly, the potential of synthetic co-cultures that combine aerobes and anaerobes is largely unexplored, and may not be limited to gas fermentation with this specific combination of microbes. Here we put forth several additional microbe pairings that could find utility for several energy-relevant applications, including bio-upgrading of C1 feedstocks (e.g., methanol, formate), valorization of waste plastics, and efficient nitrogen fixation.

Synthetic Co-Cultures are Promising for Complex Transformations, but Pairings of Aerobes and Anaerobes have been Minimally Explored

There is a rich history of using co-cultures to perform transformations that are challenging with a single microbe, such as anaerobic digestion, waste-water treatment, and lignocellulose breakdown. Native microbial consortia can have several advantages in a bioprocessing context, including stability to environmental perturbations, and resistance to contamination. Recent years have seen increased interest in the design and engineering of synthetic co-cultures, in which well-defined consortia of two or three well-characterized microbes are assembled to retain the advantages of co-culture, while i) avoiding the complexity of highly diverse natural populations, and ii) enabling targeted engineering of traits and production via metabolic engineering. Recently, it was shown that distributing a metabolic pathway for the production of a precursor to the anti-cancer medication paclitaxel between E. coli and Saccharomyces cerevisiae in a synthetic consortium resulted in higher titers than could be achieved in either microbe engineered individually for production of the target. Similarly, distributing a pathway for muconic acid production between two E. coli strains resulted in higher titer than the mono-culture, presumably by lowering the metabolic burden of heterologous protein expression on a single strain. As a final example, the syngas-fermenting Clostridium autoethanogenum was paired with Clostridium kluyveri, which is capable of chain elongation of C2 acids to C4 and C6 compounds. The anaerobic co-culture was able to produce butanol and hexanol from syngas.

Despite the growing number of successful examples in the synthetic co-culture literature, there is a paucity of studies of co-cultures containing both an aerobic and anaerobic partner. Most studies, as illustrated above, have focused either on pairs of aerobic microbes (e.g. E. coli) or pairs of anaerobes (e.g. Clostridium). A syntrophic co-culture involving the cellulose-degrading strict anaerobe Clostridium phytofermentans and S. cerevisiae was established under semi-aerobic conditions, in which the yeast protected C. phytophermentans from oxygen in exchange for soluble sugar, which it converted to ethanol. More recently, a symbiotic co-culture of Bacillus cereus (an aerobe) and Clostridium acetobutylicum (a strict anaerobe well known for ABE fermentation) was shown to produce high levels of butanol with continuous air sparging. These studies highlight the underexplored potential of microaerobic co-cultures, but have important functional differences with our system. In the first case, since S. cerevisiae produces ethanol under both anaerobic and aerobic conditions, it was unclear if the yeast was actually respiring, and therefore deriving an energy benefit from the presence of O₂. In the second case, at steady state the aerobe represented only ˜1% of the total co-culture. As this was not the butanol-producing microbe, this made no difference to their process, whereas in our system the E. coli is the main product-forming microbe.

### Oxygen Tolerance in Strict Anaerobes is Poorly Characterized

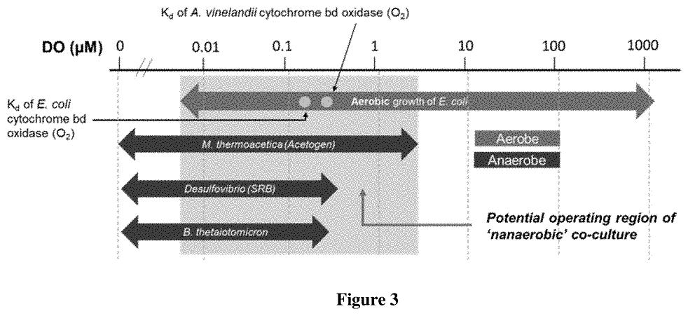

We posit that one reason anaerobe/aerobe co-cultures have not been extensively explored is because there is fundamental confusion about the level of oxygen tolerance in strict (or obligate) anaerobes. Surveying the acetogen literature, most studies of the effect of oxygen have been conducted by adding a bolus of oxygen to a culture, and observing its effects. For example, Grunden and colleagues examined growth and gene expression in C. ljungdahlii on a number of substrates after exposure to a one-time spike of 8% O₂, identifying genetic and metabolic responses to the inhibition of specific O₂-sensitive enzymes. Similar studies on other strict anaerobes have been conducted in more-or-less identical manner, reaching similar conclusions. These findings are important, because they indicate a diverse and previously unsuspected repertoire of defense mechanisms for anaerobes that encounter oxygen. However, because these studies used a one-time injection of air, and the rate of oxygen depletion was not measured, almost nothing is known about the ability of acetogens and other anaerobes to cope with a sustained, low level of O₂, which would be seen in oxygen-sparged co-culture with an aerobe. It should be noted that E. coli can grow aerobically at dissolved oxygen levels much lower than were found to enable anaerobic growth in the studies above (FIG. 3).

Our preliminary data strongly support the feasibility of microaerobic co-culture in the context of acetogen-mediated fixation of CO₂ with renewable H₂. Our analysis also suggests that other aerobe/anaerobe pairings may be highly promising for a range of other energy-relevant transformations that are currently challenging with single microbes, as described below:

Simultaneous CO₂ and N₂ Fixation to Amino Acids with Renewable H₂.

Global industrial nitrogen fixation via the Haber-Bosch process is energetically intensive, requiring high pressure and temperature, and accounting for 1.2% of total CO₂ emissions and 1-2% of worldwide energy consumption. More efficient modes of nitrogen fixation are required to lower anthropogenic CO₂ emissions while providing food for an increasing population. Biological nitrogen fixation occurs at ambient conditions and is catalyzed by nitrogenase (N₂ase). This enzyme is encoded by a diverse range of bacteria, including the hydrogenotrophic autotroph Xanthobacter autotrophicus. One attractive solution for energy-efficient, distributed N₂ fixation is therefore to couple renewable hydrogen production to simultaneous CO₂ and N₂ fixation, producing X. autotrophicus as a biofertilizer. Indeed, pioneering work from demonstrated that electrolytically produced H₂ could drive growth of X. autotrophicus with no organic carbon input under ambient conditions, with the produced biomass acted as a potent fertilizer.

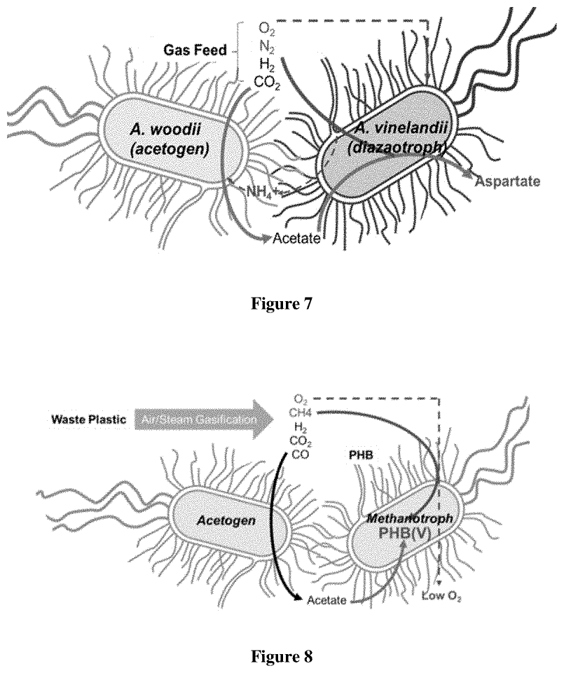

Here we propose an alternative approach based on our micro aerobic co-culture technology, using the model diazotroph Azotobacter vinelandii (FIG. 7). This microbe can fix N₂ aerobically, using a sophisticated ‘respiratory protection’ mechanism to maintain very low intracellular O₂ concentrations and protect the oxygen-sensitive nitrogenase. It also grows and fixes N₂ on acetate (its preferred carbon source), with a doubling time (t_{d}) of less than 2 hr. By contrast, because X. autotrophicus uses the ATP-inefficient Calvin cycle for CO₂ fixation, its growth is significantly slower (t_{d}=3.5 hr). The high affinity of A. vinelandii for O₂ (equal to E. coli), its ability to secrete NH₄⁺, and its ability to grow well on the end-product of acetogenic CO₂ fixation, make it an ideal candidate for microaerobic co-culture. Although some acetogens encode N₂ase, their ATP-limited lifestyle results in very slow diazotrophic growth, so N₂ fixation is best done by the aerobe. In principle, beyond simply providing biomass as fertilizer, this method could be coupled with a strain A. vinelandii metabolically engineered for the production of specific amino acids (e.g. aspartate, a DOE Top Value-Added target). This co-culture provides a renewable production platform for platform amino acids starting from entirely gaseous substrates, leveraging the high efficiency of acetogenic carbon fixation in the co-culture technology.

Production of Bioplastics from Gasified Waste Plastics.

Plastic waste affects environmental quality and ecosystem health, with the U.S. being responsible for ˜65 million metric tons in 2016. Cost-effective technologies for upcycling of plastic waste are urgently needed, as in their absence currently 75% of all plastic waste is sent to landfills. The first step in any plastic upcycling scheme is the breakdown of polymeric material into monomeric components, a process made particularly challenging by the heterogeneous nature of plastic waste. One promising approach is gasification—the oxidative conversion with air or steam into a mix of H₂, CO, methane (CH₄) and CO₂. The advantage of this approach is its feedstock flexibility; however a downside is the need to further convert the product gases. Here we put forward the concept of a co-culture of an acetogen with an aerobic methanotroph to valorize these gas mixtures (FIG. 8). The methanotroph would grow using CH₄ (which can represent 10-30% of the gas stream), ensuring an anaerobic environment for the acetogen to convert the CO, CO₂, and H₂ efficiently into acetate. Notably, some methanotrophs have been shown to co-utilize CH₄ and acetate, and others are well-known for the production of the bioplastic PHB or the more desirable copolymer PHBV at high quantities under oxygen-limited conditions. Thus the co-culture could provide a potential avenue for circular plastics production with very high carbon conversion efficiency.

High-Yield Conversion of Additional C1 Substrates.

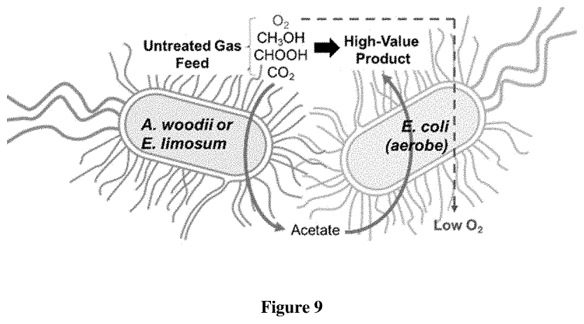

In addition to syngas mixtures, several acetogens are capable of growing on the soluble C1 feedstocks formate and methanol, such as E. limosum and Acetobacterium woodii. These substrates are increasingly attractive for bioprocessing as they can be produced renewably from CO₂, and avoid the mass transfer limitations associated with low-solubility gases. Again because of their use of the highly-efficient Wood-Ljungdahl pathway for this conversion, acetogenic methylotrophy and formatotrophy offer a combination of high yield and high productivity. Though the energetics of acetogenic methanol conversion are better than for H₂, still the anaerobic process is limited to low-value end products. Here we put forth the concept of using additional acetogens in the microaerobic co-culture to enable production of higher-value molecules from an expanded set of C1 substrates (FIG. 9).

Additional Pairs of Microbes for CO₂ Fixation

We have demonstrated the co-culture with C. ljungdahlii and E. coli, but in principle the technology could extend to any acetogen combined with any aerobic acetotroph.

Acetogens are obligate anaerobic bacteria that dwell in energy-poor environments near the very thermodynamic limit of life, making a living by using hydrogen (H₂), carbon monoxide (CO), and other reduced organic carbon compounds to convert carbon dioxide (CO₂) into acetate. An acetogen may be a bacteria that generates acetate (CH₃COO⁻) as an end product of anaerobic respiration or fermentation. This may be bacteria that perform anaerobic respiration and carbon fixation simultaneously through the reductive acetyl coenzyme A (acetyl-CoA) pathway (also known as the Wood-Ljungdahl pathway). They can produce acetyl-CoA (and from that, in most cases, acetate as the end product) from two molecules of carbon dioxide (CO₂) and four molecules of molecular hydrogen (H₂). This process is known as acetogenesis, and is different from acetate fermentation, although both occur in the absence of molecular oxygen (O₂) and produce acetate.

Acetogenic bacteria may be of the genus Clostridium, Moorella, Ruminococcus, Acetobacterium, Eubacterium, Butyribacterium, Blautia, Alkalibaculum, Sporomusa, or Oxobacter. Acetogenic bacteria may be Clostridium ljungdahlii, Clostridium autoethanogenum, Eubacterium limosumi, Acetobacterium woodii, Butyribacterium methylotrophicum, Blautia hydrogenotrophica, Clostridium carboxidivorans, Clostridium drakei, Clostridium coskatii, Alkalibaculum bacchi, Blautia product, Sporomusa ovata, Sporomusa silvacetica, Sporomusa sphaeroides, Oxobacter pfennigii, Clostridium aceticum, Clostridium formicaceticum, Clostridium tetanomorphum, Moorella thermoacetica, Moorella thermoautotrophica, Ruminococcus productus, or Acetobacterium woodii.

An acetotroph is a microorganism that metabolizes acetate, typically yielding a product such as methane and carbon dioxide.

An anaerobic organism or anaerobe is any organism that does not require molecular oxygen for growth. It may react negatively or even die if free oxygen is present. In contrast, an aerobic organism (aerobe) is an organism that requires an oxygenated environment.

Examples of aerobic acetotroph are Escherichia coli, Vibrio natriegens, or Yarrowia lipolytica.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

As used herein, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The term “comprise” is generally used in the sense of include, that is to say permitting the presence of one or more features or components. Wherever embodiments, are described herein with the language “comprising,” otherwise analogous embodiments described in terms of “consisting of,” and/or “consisting essentially of” are also provided.

The term “microaerobic environment” generally means an environment where the dissolved oxygen concentration in the growth media is substantially reduced compared to the equilibrium concentration in normal atmosphere.

### EXAMPLES

The invention now being generally described, it will be more readily understood by reference to the following examples that are included merely for purposes of illustration of certain aspects and embodiments of the present invention, and are not intended to limit the invention.

### Example 1. Methods

### Microbial Strains

Clostridium ljungdahlii PETC was obtained from the American Type Culture Collection (ATCC #55383). Escherichia coli MG1655(DE3) was a gift from G. Stephanopoulos (MIT).

### Cell Cultivation

C. ljungdahlii was grown as described in B. M. Woolston, et al., Rediverting Carbon Flux in Clostridium Ljungdahlii Using CRISPR Interference (CRISPRi), Metabolic Engineering 48, 243 (2018). For general cultivation, C. ljungdahlii was grown anaerobically on rich medium (YTF), which contains per liter: yeast extract 10 g, Bacto tryptone 16 g, NaCl 4 g, fructose 5 g, resazurin 0.5 mL of 0.2% stock solution, cysteine-HCl 0.3 g). For bioreactor experiments with rich medium, the fructose was replaced with the same quantity of xylose and glycerol, as described below. For experiments in minimal medium, cells were grown in PETC 1754 medium (per liter: NH₄Cl 1 g, KCl 0.1 g, MgSO₄.7H₂O 0.2 g, NaCl 0.8 g, KH₂PO₄ 0.1 g, CaCl₂.2H₂O 20 mg, yeast extract 1 g, ATCC trace minerals (MD-TMS) 10 mL, ATCC vitamins (MD-VS) 10 mL, NaHCO₃ 2 g, resazurin 0.5 mL of 0.2% stock solution, cysteine-HCl 0.3 g) supplemented with 5 g L-1 xylose or fructose for heterotrophic growth or with 20 psig of 80:20 H₂:CO₂ headspace for autotrophic growth. For construction of mutant strains and cloning of plasmids E. coli was cultivated in LB medium according to standard practices, supplemented with appropriate antibiotics (chloramphenicol, 25 ug/mL; kanamycin, 50 ug/mL; spectinomycin, 100 ug/mL). All anaerobic manipulations were performed in aa anaerobic chamber (Anaerobe Systems AS580) with an atmosphere of 5% H₂, 10% CO₂, balance N₂.

### Bioreactor Experiments

Bioreactor experiments were carried out in a 1 L Eppendorf BioFlow 120 reactor, with a working volume of 500 mL. Agitation rate was 250 rpm, with sparge gas rate of 250 mL/min. The reactor was temperature-controlled to 37 C, and equipped with a condenser with cooling water flow at 16 C to minimize evaporative losses of volatile products and water. pH control was accomplished with online pH probe and a 5M NaOH base feed. Dissolved oxygen was measured by DO probe, with two-point calibration using sparged air (100%) and N₂ (0%). Redox was measured by probe, calibrated in a saturated solution of quinhydrone. E. coli was typically inoculated at a dilution of 1:100 from overnight culture in the same media, whereas C. ljungdahlii was inoculated with a 1:10 dilution. Gas mixing was accomplished using a custom-built mass flow controller (MFC)-based system (Alicat, USA), in which individual pure gas streams were flowed at specified rates into a mixed stream through a series of tees, and then into a holding cylinder, from which the mixed gas was metered out through a flashback arrestor to the bioreactor via another MFC. Growth was measured by optical density using a SpectraMax QuickDrop spectrophotometer.

### Product Quantification

Extracellular metabolites were analyzed using an Waters Alliance HPLC system with a G1362A RID detector with isocratic flow of 0.1% formic acid (0.7 mL min-1, 50° C.) through a BioRad Aminex HPX-87H ion exchange column (300 mm×7.8 mm). 500 μL culture was withdrawn and filtered through 0.2 μm syringe filter into HPLC vials, and 10 μL injected into the HPLC. Product identity and concentration was confirmed by comparison to standard curves generated from pure compounds.

E. coli Genetic Manipulations

The ΔdapA and ΔxylA strains were constructed using previously established techniques based on the lambda Red and CRISPR-Cas9 systems. All mutants were verified via colony PCR using primers targeting the region spanning the deleted genes, such that WT and mutant strains would produce different sized bands. The 3HB production plasmids were constructed using Gibson Assembly (NEB Hi-Fi Mastermix) according to the manufacturer's protocols, using genes phaA and phaB amplified from the chromosome of C. necator using Q5 Polymerase (NEB), and pct from either Clostridium beijerinckii or Clostridium propionicum custom synthesized as gBlocks by IDT DNA. The genes were expressed under control of the IPTG-inducible T7 promoter on the commercial plasmid pACYC-Duet (Novagen), which we modified to additionally express the deleted dapA gene as an operon with the chloramphenicol resistance gene catP.

### Example 2. C. ljungdahlii and E. coli Co-Culture in O₂-Sparged Bioreactors

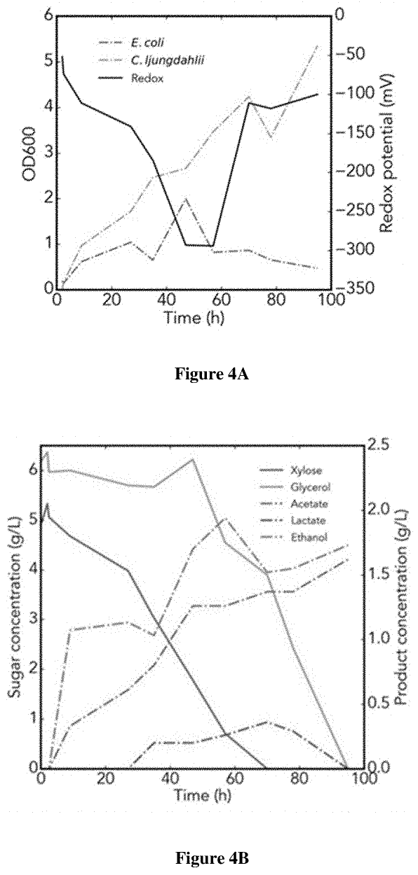

We have demonstrated the successful co-cultivation of the model acetogen Clostridium ljungdahlii and E. coli in bioreactors sparged with 1% O₂. To the best of our knowledge, this is the first evidence of the sustained growth of an acetogen in a microaerobic environment. We first examined the system heterotrophically, using the orthogonal substrates glycerol (not consumed by C. ljungdahlii) and xylose (not consumed by the ΔxylA E. coli strain we generated) in rich medium. The reactor was inoculated first with E. coli, with a gas stream of 10% air, 70% N₂, and 20% CO₂. DO was monitored until it dropped below the probe detection limit (˜0.5%), then C. ljungdahlii was inoculated. Total growth was monitored by OD₆₀₀, and the abundance of each microbe determined by qPCR targeting previously validated genes. Product formation and substrate consumption were determined by HPLC-RID. As shown in FIG. 4, C. ljungdahlii grew rapidly even with continual sparging, reaching a final OD of ˜5. Within approximately 10 hours, C. ljungdahlii had become the dominant microbe in the reactor, and the redox potential dropped to a level typical of Clostridial fermentations. Additionally, acetate and ethanol, typical products of C. ljungdahlii metabolism, were detected in the broth. Xylose and glycerol were both consumed, indicating metabolic activity of both microbes. Importantly, when acetogens grow on sugar substrates, the Wood-Ljungdahl pathway and its oxygen-sensitive enzymes are still required. Additionally, conversion of pyruvate to acetyl-CoA (the precursor to acetate) uses the highly oxygen-sensitive enzyme pyruvate:ferredoxin oxidoreductase (PFOR). Thus, the robust growth in this experiment demonstrates the successful protection of all the enzymes necessary for autotrophic growth.

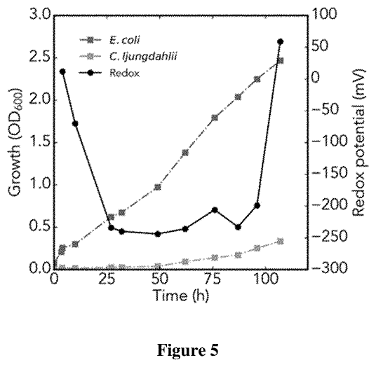

We next examined whether the co-culture could be established in a minimal medium, which would be cheaper for a production scenario. Using the same reactor operation strategy, we again saw simultaneous growth of both microbes (FIG. 5). Interestingly, the relative abundances were very different, with E. coli accounting for most of the biomass under these conditions. In both these experiments, we monitored the redox potential of the culture, noticing a significant drop from +50 mV to −250 mV, which is typical for Clostridial fermentation, and signifies the ability of C. ljungdahlii to establish a desirable redox environment despite the oxygen feed. Importantly, in minimal medium, the consumption of glycerol by E. coli requires respiration. Thus this experiment goes beyond previously any aerobic/anaerobic co-cultures, demonstrating that E. coli is benefitting energetically from the oxygen, highlighting the potential for energy-intensive product synthesis. It also shows that we can tune the relative abundance of each microbe by manipulating the media compositions, paving the way to optimizing the platform for production.

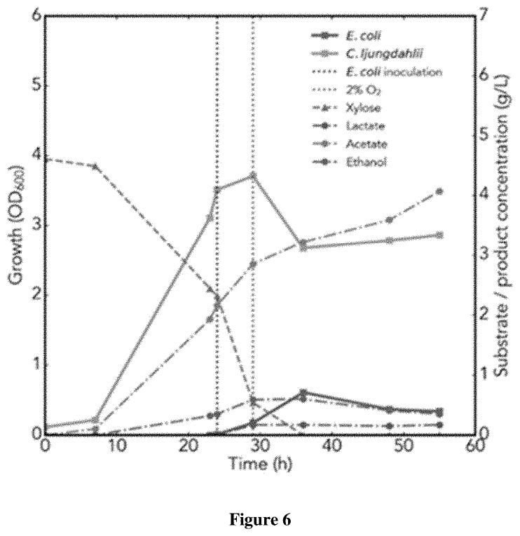

We next asked whether we could reverse the order of the inoculation. We therefore initiated the reactor with C. ljungdahlii and a gas feed containing no oxygen, then added oxygen to the feed and inoculated E. coli after 24 hours of growth (FIG. 6). Again, we saw concurrent growth of both microorganisms as evidenced by density increases and xylose consumption between 24 and 30 hours. In this case, the short window of co-culture growth was due to exhaustion of the xylose by C. ljungdahlii. We hypothesize performance could have been improved by either earlier E. coli inoculation to prevent complete consumption of media components by C. ljungdahlii, or by further xylose additions to keep C. ljungdahlii growth going. However, the main important results were i) that the co-culture approach is robust enough to enable reversing the order of inoculation, and ii) the co-culture worked at a gas feed of 10% air (2% oxygen, which was double the level we had used previously). Collectively, these experiments demonstrate the feasibility of acetogen/aerobe co-culture.

### Example 3. Demonstration with CO₂

Experiments are conducted to establish growth of the co-culture on gases, rather than sugars. These experiments are performed similarly to those in FIGS. 4 and 6. Briefly, in cases where E. coli is inoculated first, E. coli is grown in rich medium containing a small amount of acetate (˜1-2 g/L) with a gas mix typically of 70% H₂, 20% CO₂, 5% N₂ and 5% air. Once the DO reads 0 in the bioreactor, C. ljungdahlii (pre-grown on rich medium supplemented with an 80/20 H₂/CO₂ atmosphere at 20 psi) is inoculated into the reactor. Growth is monitored by OD measurements, production via HPLC, and gas consumption monitored by online GC. qPCR is used as above to determine the relative abundance of each microbe. In cases where the inoculation order is reversed, C. ljungdahlii pre-grown as above is inoculated into the reactor containing rich media, with an anaerobic gas feed of 70% H₂, 20% CO₂, 10% N₂. Once robust growth is established, E. coli pre-grown on acetate is inoculated, and the gas composition adjusted to 70% H₂, 20% CO₂, 5% N₂ and 5% air. Reactor progress is monitored as above. In these experiments, the consumption of H₂ and CO₂ in the reactor as determined by online GC analysis provides evidence that the C. ljungdahlii is growing autorophically, and not just using additional nutrients (e.g. yeast extract) that are available in the rich media.

### Example 4. Demonstration of Product Formation

The technology could be used to produce any heterologous product that can be engineered into E. coli. The technology is demonstrated with 3-hydroxyburyate, a value-added chemical with a range of uses. To develop an E. coli strain capable of producing 3-hydroxybutyric acid (3HB) from acetate, we constructed plasmids expressing all three of the necessary genes encoding the enzymes in the 3HB pathway. Typically, stable replication of plasmids in their host is accomplished by engineering an antibiotic resistance gene into the plasmid, then treating the culture with the corresponding antibiotic to ensure that cells need the plasmid to survive. However, this approach is challenging in a co-culture as the partner microbe (C. ljungdahlii) does not have the resistance gene. To avoid this problem, we used a diaminopimelic acid (DAP) auxotrophy strategy previously demonstrated to enable antibiotic-free selection of plasmids in E. coli in a variety of media. DAP is a component of the Gram-negative cell wall which is not found in most media. E. coli produces DAP by the enzyme encoded by the dapA gene. To make E. coli dependent on the 3HB plasmid, the dapA gene was removed from the E. coli chromosome via CRISPR-Cas9 and placed on the plasmid expressing the 3HB genes. We then successfully confirmed that E. coli MG1655 ΔdapA ΔxylA exhibited no growth at all in the absence of DAP, but that when transformed with the dapA-containing plasmid, growth was restored in without DAP supplementation. This provided us with a plasmid and E. coli strain that we could use for expression of the 3HB genes in the co-culture system without interfering with C. ljungdahlii.

We first attempted to use the 3HB production pathway demonstrated by H.-C. Tseng et al. (Applied and Environmental Microbiology 75, 3137 (2009)). This pathway used phaA and phaB native to R. eutropha and tesB found in E. coli. These genes were PCR amplified from their respective sources and inserted following the T7 promoter in the dapA plasmid. We tested the 3HB production capability of each of these strains carrying these plasmids in PETC and YT media supplemented with acetate, glycerol, or glucose as carbon sources. We were surprised to find that 3HB was only produced by glucose-fed cultures. This was disappointing, as we needed to ensure production with acetate as the carbon source for use in the co-culture system. A thorough review of the literature led us to a recent paper by Fei et al (Bioresource Technology 336, 125323 (2021)), in which it was reported that a pct gene from Clostridium beijerinckii was able to produce 3HB from acetate whereas the tesB gene that we originally used was unable to. We thus generated two new plasmids where tesB was swapped with a pct from either C. beijerinckii or C. propionicum. Gratifyingly, when these plasmids were transformed into the DAP auxotroph strain, we successfully measured conversion of acetate to 3HB via LCMS, with a yield of 0.25 g 3HB per g acetate consumed. Thus, we successfully accomplished our aim of generating an E. coli strain that could convert acetate to 3HB in a co-culture environment. This strain is tested in the co-culture system.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of culturing an anaerobic bacteria, comprising culturing in a microaerobic environment the anaerobic bacteria and an aerobic microorganism.

2. The method of claim 1, wherein the microaerobic environment does not require gas pre-treatment to remove trace O₂.

3. The method of claim 1, wherein the microaerobic environment comprises O₂, CO, H₂, and CO₂.

4. The method of claim 1, wherein the microaerobic environment comprises O₂, N₂, H₂, and CO₂.

5. The method of claim 1, wherein the microaerobic environment comprises O₂, CH₄, H₂, CO₂, and CO.

6. The method of claim 1, wherein the microaerobic environment comprises O₂, CHOOH, CHOOH, and CO₂.

7. (canceled)

8. The method of claim 1, wherein the anaerobic bacteria are acetogenic bacteria.

9. (canceled)

10. The method of claim 8, wherein the acetogenic bacteria are of the genus Clostridium, Moorella, Ruminococcus, Acetobacterium, Eubacterium, Butyribacterium, Blautia, Alkalibaculum, Sporomusa, or Oxobacter.

11. The method of claim 10, wherein the acetogenic bacteria are Clostridium ljungdahlii, Clostridium autoethanogenum, Eubacterium limosumi, Acetobacterium woodii, Butyribacterium methylotrophicum, Blautia hydrogenotrophica, Clostridium carboxidivorans, Clostridium drakei, Clostridium coskatii, Alkalibaculum bacchi, Blautia product, Sporomusa ovata, Sporomusa silvacetica, Sporomusa sphaeroides, Oxobacter pfennigii, Clostridium aceticum, Clostridium formicaceticum, Clostridium tetanomorphum, Moorella thermoacetica, Moorella thermoautotrophica, Ruminococcus productus, or Acetobacterium woodii.

12. The method of claim 1, wherein the aerobic microorganism is an aerobic acetotroph microorganism.

13. The method of claim 12, wherein the aerobic acetotroph microorganism converts the acetate to a product.

14. The method of claim 13, wherein the product is ethanol, 2,3-butanediol (2,3-BDO), bioproducts 3-hydroxybutyrate (3HB), butanol, farnesene, acetic acid, butyric acid, methane, or an amino acid.

15. (canceled)

16. The method of claim 1, wherein the aerobic microorganism is Escherichia coli, Vibrio natriegens, or Yarrowia lipolytica.

17. The method of claim 1, wherein the aerobic microorganism is of the genus Azotobacter.

18. The method of claim 17, wherein the aerobic microorganism is Azotobacter vinelandii or Azotobacter chroococcum.

19. The method of claim 1, wherein the aerobic microorganism is a methanotroph microorganism.

20. The method of claim 19, wherein the aerobic microorganism is a Type II methanotroph bacteria.

21. The method of claim 20, wherein the methanotroph bacteria are of the genus Methylocystis.

22. (canceled)

23. A method of producing a product, comprising culturing in a microaerobic environment an anaerobic bacteria and an aerobic microorganism.

24. A method of syngas fermentation, comprising culturing in a microaerobic environment an anaerobic bacteria and an aerobic microorganism.

25.-46. (canceled)
